(19) United States
(12) Patent Application Publication
(10) Pub. No.: US 2006/0089678 A1
(43) Pub. Date: Apr. 27, 2006

(54) Technique for blood pressure regulation

(76) Inventor: Alon Shalev, Ra'anana (IL)

Correspondence Address: DARBY & DARBY P.C., P. O. BOX 5257, NEW YORK, NY, 10150-5257, US

(21) Appl. No.: 10/507,703

(22) PCT Filed: Mar. 13, 2003
(86) PCT No.: PCT/IL03/00215

§ 371 Date: Jun. 9, 2005

(30) Foreign Application Priority Data
Mar. 14, 2002 (US) 60364829

(51) Int. Cl. A61N 1/365

(52) U.S. Cl. 607/23

(57) ABSTRACT

An implantable device (20) uses the carotid baroreflex in order to control systemic blood pressure. The implant includes sampling and pulse stimulation electrodes (44) preferably located on the carotid sinus nerve branch of the glossopharyngeal nerve, adjacent and distal to the carotid sinus baroreceptors. The stimulators have an external control unit, which communicates with the implant for determining appropriate operational parameters, and for retrieving telemetry information from the device's data bank. Typically two internal devices are implanted, one at each side of the patient's neck.

### BACKGROUND OF THE INVENTION

1. Field of the Invention

This invention relates to medical apparatus for the treatment of hypertension. More particularly this invention relates to an implant that uses the carotid baroreflex in order to control systemic blood pressure.

2. Description of the Related Art

Cardiovascular Regulation of Blood Pressure.

In human physiology, several negative feedback systems control blood pressure by adjusting heart rate, stroke volume, systemic vascular resistance and blood volume. Some allow rapid adjustment of blood pressure to cope with sudden changes such as the drop in cerebral blood pressure when rising up. Others act more slowly to provide long-term regulation of blood pressure. Even if blood pressure is steady, there may be a need to change the distribution of blood flow, which is accomplished mainly by altering the diameter of arterioles.

Groups of neurons scattered within the medulla of the brain stem regulate heart rate, contractility of the ventricles, and blood vessel diameter. As a whole, this region is known as the cardiovascular center, which contains both a cardiostimulatory center and a cardioinhibitory center. The cardiovascular center includes a vasomotor center, which includes vasoconstriction and vasodilatation centers that influence blood vessel diameter. Since these clusters of neurons communicate with one another, function together, and are not clearly separated anatomically, they are usually taken as a group.

The cardiovascular center receives input both from higher brain regions and from sensory receptors. Nerve impulses descend from higher brain regions including the cerebral cortex, limbic system and hypothalamus to affect the cardiovascular center. The two main types of sensory receptors that provide input to the cardiovascular center are baroreceptors and chemoreceptors. Baroreceptors are important pressure-sensitive sensory neurons that monitor stretching of the walls of blood vessels and the atria. Chemoreceptors monitor blood acidity, carbon dioxide level and oxygen level.

Output from the cardiovascular center flows along sympathetic and parasympathetic fibers of the autonomic nervous system. Sympathetic stimulation of the heart increases heart rate and contractility. Sympathetic impulses reach the heart via the cardiac accelerator nerves. Parasympathetic stimulation, conveyed along the vagus nerves, decreases heart rate. The cardiovascular center also continually sends impulses to smooth muscle in blood vessel walls via sympathetic fibers called vasomotor nerves. Thus autonomic control of the heart is the result of opposing sympathetic (stimulatory) and parasympathetic (inhibitory) influences. Autonomic control of blood vessels, on the other hand, is mediated exclusively by the sympathetic division of the autonomic nervous system.

In the smooth muscle of most small arteries and arterioles, sympathetic stimulation causes vasoconstriction and thus raises blood pressure. This is due to activation of alpha-adrenergic receptors for norepinephrine and epinephrine in the vascular smooth muscle. In skeletal muscle and the heart, the smooth muscle of blood vessels displays beta-adrenergic receptors instead, and sympathetic stimulation causes vasodilatation rather than vasoconstriction. In addition, some of the sympathetic fibers to blood vessels in skeletal muscle are cholinergic; they release acetylcholine, which causes vasodilatation.

Neural Regulation of Blood Pressure.

Nerve cells capable of responding to changes in pressure or stretch are called baroreceptors. Baroreceptors in the walls of the arteries, veins, and right atrium monitor blood pressure and participate in several negative feedback systems that contribute to blood pressure control. The three most important baroreceptor negative feedback systems are the aortic reflex, carotid sinus reflex and right heart reflex.

The carotid sinus reflex is concerned with maintaining normal blood pressure in the brain and is initiated by baroreceptors in the wall of the carotid sinus. The carotid sinus is a small widening of the internal carotid artery just above the bifurcation of the common carotid artery. Any increase in blood pressure stretches the wall of the aorta and the carotid sinus, and the stretching stimulates the baroreceptors. The carotid sinus nerve, which is an afferent nerve tract that originates in the carotid sinus baroreceptors, converges with the glossopharyngeal nerve, passes through the jugular foramen, reaches the rostral end of the medulla, and continues to the cardiovascular center.

When an increase in aortic or carotid artery pressures is detected in this manner, the cardiovascular center responds via increased parasympathetic discharge in efferent motor fibers of the vagus nerves to the heart, and by decreased sympathetic discharge in the cardiac accelerator nerves to the heart. The resulting decreases in heart rate and force of contraction lower cardiac output. In addition, the cardiovascular center sends out fewer sympathetic impulses along vasomotor fibers that normally cause vasoconstriction. The result is vasodilatation, which lowers systemic vascular resistance.

Carotid Sinus Baroreceptors.

It has been demonstrated that there are two functionally different carotid sinus baroreceptors, where each type may play a different role in the regulation of blood pressure. Reference is now made to FIG. 1A, which is a plot of baroreceptor activity, measured on the ordinate as pulses or spikes per second against carotid sinus pressure on the abscissa, measured in mm Hg.

Type I baroreceptors are characterized by a discontinuous hyperbolic transduction curve 10. Specifically, the electrical discharge pattern of these baroreceptors is such that, until a threshold carotid sinus pressure has been achieved, no signal is produced. However, when the carotid sinus pressure reaches the threshold, type I baroreceptor discharge commences abruptly, with an initial firing rate of about 30 spikes per second. Saturation occurs at about 200 mm Hg, at which the firing rate saturates at about 50 spikes per second.

The nerve fibers connected to these types of baroreceptors are mostly thick, myelinated type A-fibers. Their conduction velocity is high, and they start firing at a relatively low threshold current (i.e., they have high impedance).

The above characteristics for the type I baroreceptors suggest that they are involved in the dynamic regulation of arterial blood pressure, regulating abrupt, non-tonic changes in blood pressure.

Type II baroreceptors are pressure transducers that are characterized by a continuous transduction curve 12. Specifically, the electrical discharge pattern of these baroreceptors is such that they transmit impulses even at very low levels of arterial blood pressure. Consequently, there is no defined threshold for type II baroreceptors. The typical firing rate of type II baroreceptors in a normotensive individual is about five spikes per second. At a carotid sinus pressure of about 200 nm Hg, the firing rate saturates at about 15 spikes per second.

The nerve fibers connected to type II baroreceptors are either thin, myelinated type A fibers, or unmyelinated type C fibers. Their conduction velocity is low and, when stimulated experimentally, they start firing at a relatively high threshold current, due to their relatively low impedance.

The above characteristics of type II baroreceptors suggest that they are involved in the tonic regulation of arterial blood pressure, and that they play a role in the establishment of baseline blood pressure (i.e., diastolic blood pressure).

Resetting Mechanism

Referring again to FIG. 1A, “resetting” is defined as a shift in the response curve of a baroreceptor, marked by shifts in the curve 10 along the abscissa, in the same direction as the change in intravascular pressure to which the baroreceptor is exposed. In animal studies, type I baroreceptors, but not type II baroreceptors, were found to reset in response to acute changes in blood pressure. This evidence supports the notion that the two types of baroreceptors have different functional roles in the regulation of arterial blood pressure. Thus, a right-shifted curve 14 represents type I baroreceptor activity that would result from an abrupt elevation of arterial blood pressure, wherein the subject's baseline activity level is shown by the curve 10.

Modulation of Baroreceptor Activity

The baroreceptive endings of the carotid sinus nerve and the aortic depressor nerve are the peripheral terminals of a group of sensory neurons with their soma located in the petrosal and nodose ganglia. The endings terminate primarily in the tunica adventitia of the carotid sinus and aortic arch. When stretched, they depolarize. Action potentials are consequently triggered from a spike-initiating zone on the axon near the terminal. The action potentials travel centrally to the nucleus tractus solitarius in the medulla. There, the sensory neurons synapse with a second group of central neurons, which in turn transmit impulses to a third group of efferent neurons that control the parasympathetic and sympathetic effectors of the cardiovascular system.

The vascular structure of the carotid sinus and aortic arch determines the deformation and strain of the baroreceptor endings during changes in arterial pressure. For this reason, structural changes in the large arteries and decreased vascular distensibility, also known as compliance, are often considered the predominant mechanisms responsible for decreased baroreflex sensitivity and resetting of baroreceptors, which occur in hypertension, atherosclerosis, and aging.

The process of mechanoelectrical transduction in the baroreceptors depends on two components: (1) a mechanical component, which is determined by the viscoelastic characteristics of coupling elements between the vessel wall and the nerve endings, and (2) a functional component, which is related to (a) ionic factors resulting from activation of channels or pumps in the neuronal membrane of the baroreceptor region, which alter current flow and cause depolarization resulting in the generation of action potentials, and (b) paracrine factors released from tissues and cells in proximity to the nerve endings during physiological or pathological states. These cells include endothelial cells, vascular muscle cells, monocytes, macrophages, and platelets. The paracrine factors include prostacyclin, nitric oxide, oxygen radicals, endothelin, platelet-derived factors, and other yet unknown compounds. Extensive animal studies conducted in the 1990s support the concept that the mechanoelectrical transduction in baroreceptor neurons occurs through stretch-activated ionic channels, whose transduction properties are affected by the aforementioned factors.

There exists evidence indicating a dependency of the baroreflex on the temporal characteristics of discharges in the cardiovascular afferent fibers. The coupling of afferent baroreceptor activity with the central group of neurons leads to inhibition of sympathetic nerve activity. This coupling was examined by determining the relationship between afferent baroreceptor activity and efferent sympathetic nerve activity measured simultaneously.

Sustained inhibition of sympathetic nerve activity is not simply a function of baroreceptor spike frequency, but depends on the phasic burst pattern, with on and off periods during systole and diastole, respectively. Sympathetic nerve activity is disinhibited, because of what may be viewed as a “central adaptation,” during nonpulsatile, nonphasic baroreceptor activity. It is not actually the pulse pressure that is important in sustaining sympathetic inhibition, but rather the magnitude of pulsatile distension of the carotid sinus and the corresponding phasic baroreceptor discharge. One would predict that a decrease in large artery compliance, as might occur in chronic hypertension or atherosclerosis, could result in a decrease in pulsatile distension of the carotid sinus and a blunting of the phasicity of baroreceptor input. There is progressive loss of the buffering capacity of the baroreflex because of central adaptation.

It has been shown experimentally that the reflex inhibition of sympathetic nerve activity is most pronounced at lower frequencies of pulsatile pressure and during bursts of baroreceptor activity (between 1 and 2 Hz). When the burst or pulse frequency exceeded 3 Hz, there is known to be a significant disinhibition of sympathetic nerve activity, despite a maintained high level of total baroreceptor spike frequency per unit time. Thus, at very rapid pulse rates the efficiency of afferent-efferent coupling is reduced.

In a study conducted using young (1 year old) and old (10 years old) beagle dogs, it was found that the reflex inhibition of sympathetic nerve activity after a rise in carotid sinus pressure was maintained in the young but was very transient in the old dogs. The “escape” of sympathetic nerve activity from baroreflex inhibition occurred in the old dogs despite a maintained increase in afferent baroreceptor activity. Thus, the major defect in the baroreflex with aging may not be a structural vascular defect or an impaired baroreceptive process, but rather a central neural defect in the afferent-efferent coupling.

It is proposed in U.S. Pat. No. 4,201,219 to employ a neurodetector device in order to generate pulsed electrical signals. The frequency of the impulses is utilized to pace the heart directly in order to modify the cardiac rate. This approach has not been generally accepted, as there were serious technical difficulties with the implantation, and the reliability of the apparatus.

In U.S. Pat. No. 3,650,277 it is proposed to treat hypertension by stimulating afferent nerve paths from the baroreceptors of a patient, in particular the nerves from the carotid sinus. Short electrical pulses are used during a limited period of the cardiac cycle. It is necessary to synchronize a pulse generator to the heart activity of the patient, either by measuring electrical activity of the heart, or by using a transducer that is capable of measuring instantaneous blood pressure.

Another attempt at simulating the baroreceptor reflex is disclosed in U.S. Pat. No. 4,791,931, wherein a pressure transducer and a cardiac pacemaker are implanted. The pacing rate is variable and is responsive to arterial pressure.

### SUMMARY OF THE INVENTION

It is an object of some aspects of the present invention to provide an improved method for controlling blood pressure in a living body by stimulation of nerves carrying carotid sinus baroreceptor impulses.

It is another object of some aspects of the present invention to provide a simplified implantable device for controlling blood pressure in a living body by neural stimulation, responsive to static measurements of a cardiovascular parameter, such as blood pressure.

It is yet another object of some aspects of the present invention to provide an implantable device, which autonomously controls blood pressure in a living body using neural stimulation without recourse to blood pressure transducers.

These and other objects of the present invention are attained by at least one implant that uses the carotid baroreflex in order to control systemic blood pressure. The implant includes sampling and pulse stimulation electrodes, located on the glossopharyngeal nerve, adjacent and distal to the carotid sinus baroreceptors. The stimulators of the implant have an external control unit, which communicates with the implants for determining appropriate operational parameters, such as pulse rate, pulse intensity, pulse spacing, increase percentage, and for retrieving telemetry information from the device's data bank. Typically two internal devices are implanted, one at each side of the patient's neck.

Principles of Operation

In a preferred embodiment of the present invention, the sensed component of the carotid baroreflex that is generated by type II baroreceptors is modulated in order to regulate tonic blood pressure. This is accomplished by exploiting the fact that the two types of baroreceptor discharge patterns can be considered to be non-overlapping in terms of discharges per unit time.

Simulating higher baroreceptor discharge rates is achieved in accordance with a preferred embodiment of the invention by adding pulsatile activity to the afferent baroreceptors' neural tract at a rate that falls within the typical regime of operation for the type II baroreceptors, e.g., from about 1 to 15 pulses per second. Implementation of this principal of operation primarily simulates enhanced activity of type II baroreceptors, and, correspondingly, simulates higher diastolic blood pressure. The desired result of the simulation of higher diastolic blood pressure is a vascular response that reduces the diastolic blood pressure.

Typically, the pulses applied to the neural tract to simulate enhanced type II activity are applied at a rate significantly slower than the range of firing rates associated with type I baroreceptors. The added pulses are thus expected to have at most a negligible effect on dynamic blood pressure regulation.

A device according to a preferred embodiment of the invention is synchronized to the patient's heartbeat, by continuously monitoring the neural activity of the carotid sinus baroreceptor nerve, which varies during different portions of the cardiac cycle. Signal detection and processing are performed, for example, tracking a moving-average of integrated neural signal power, and peak detection. Synchronization with the cardiac cycle facilitates an accurate simulation of the baroreceptor discharge pattern, which results in effective blood pressure regulation. In a preferred embodiment, the pulses are applied at least in part during diastole, i.e., when type II discharge naturally predominates and type I discharge is reduced or absent.

### BRIEF DESCRIPTION OF THE DRAWINGS

For a better understanding of these and other objects of the present invention, reference is made to the detailed description of the invention, by way of example, which is to be read in conjunction with the following drawings, wherein:

FIGS. 1A and 1B are plots of baroreceptor activity versus carotid sinus pressure, FIG. 1B showing a level of signal application in accordance with a preferred embodiment of the present invention;

FIG. 2 is a block diagram of an arrangement for blood pressure control in accordance with a preferred embodiment of the invention;

FIG. 3 is an anatomic drawing illustrating aspects of the arrangement shown in FIG. 2;

FIG. 4 is a schematic diagram illustrating the arrangement shown in FIG. 2 in further detail;

FIG. 5 is a flow chart illustrating a method of operation of the arrangement for regulating blood pressure according to a preferred embodiment of the invention;

FIG. 6 is a schematic diagram of an arrangement for controlling blood pressure in accordance with an alternate embodiment of the invention;

FIG. 7 is a detailed block diagram of an implanted device of the embodiment shown in FIG. 6;

FIG. 8 is a block diagram of an external controller of the embodiment shown in FIG. 6;

FIG. 9 illustrates plots of type II baroreceptor activity against carotid sinus pressure in physiologic and hypertensive states; and

FIG. 10 is a flow chart illustrating a method of operation of the arrangement for blood pressure regulation shown in FIG. 6.

### DESCRIPTION OF THE PREFERRED EMBODIMENT

In the following description, numerous specific details are set forth in order to provide a thorough understanding of the present invention. It will be apparent however, to one skilled in the art that the present invention may be practiced without these specific details. In other instances well known circuits, control logic, and the details of computer program instructions for conventional algorithms and processes have not been shown in detail in order not to unnecessarily obscure the present invention.

### First Embodiment

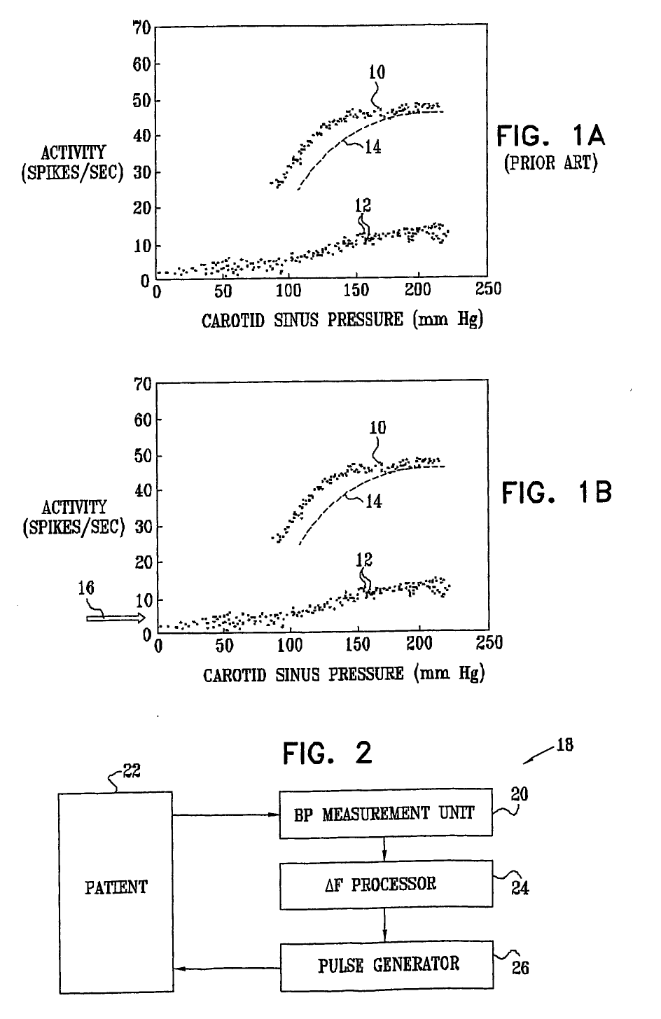

Reference is now made to FIGS. 1B and 2. FIG. 1B is a graph of recorded baroreceptor activity versus carotid sinus pressure, showing a level of signal application to facilitate blood pressure regulation, in accordance with a preferred embodiment of the present invention. FIG. 2 is a high level block diagram of an arrangement for blood pressure control, which is constructed and operative in accordance with a preferred embodiment of the invention. In an arrangement 18, a blood pressure measurement device 20 is connected to a patient 22. The blood pressure measurement device 20 can be a conventional arm-cuff sphygmomanometer, which intermittently provides input information. In stable situations, blood pressure information could be recorded relatively infrequently, e.g., daily or weekly, while in other patients, the measurement frequency could be higher, and may be adjusted. It is an advantage of this embodiment of the invention that autonomous automatic mechanical blood pressure measurement devices are rendered unnecessary. These devices are complicated, often unreliable, and have proven to be a limiting factor in the utility of earlier hypertension control techniques. Techniques described hereinbelow are preferably additionally utilized, in order to obtain real-time measurements of the patient's diastolic and/or systolic blood pressure.

The information obtained from the blood pressure measurement device 20 is provided to a processor 24, which can be realized as a simple microprocessor. The processor 24 determines an effective baroreceptor discharge rate required to compensate the blood pressure of the patient 22. A target diastolic and/or systolic blood pressure value and typical type II and/or type I baroreceptor response data are stored in a memory of the processor 24.

The output of the processor 24 is coupled to a pulse generator 26, which is preferably implanted in the patient 22 using known techniques. The pulse generator 26 can be the devices that are disclosed in U.S. Pat. Nos. 3,522,811 and 5,154,172. Other impulse generators for neural stimulation are known, as well. For example, an implantable neurostimulator, suitable for the pulse generator 26, is the Model 101 NCP Pulse Generator, available from Cyberonics, Inc., 16511 Space Center Blvd., Suite 600, Houston, Tex. U.S.A. 77058. In some embodiments the processor 24 and the pulse generator 26 may be integrated.

Preferably, as described in greater detail hereinbelow, the pulse generator 26 generates pulses at a rate such as that indicated by a rate designator 16 (FIG. 1B), such that the applied pulses are conveyed towards the patient's brain along with pulses naturally generated by type II baroreceptors. In this manner, the patient's natural blood pressure regulation apparatus interprets the combination of the natural and the applied pulses to indicate a higher diastolic blood pressure than actually exists, and responds more forcefully to lower the diastolic blood pressure. Typically, the rate at which the pulse generator 26 applies pulses is gradually reduced in response to indications by the blood pressure measurement device 20 that the patient's blood pressure is approaching a desired value.

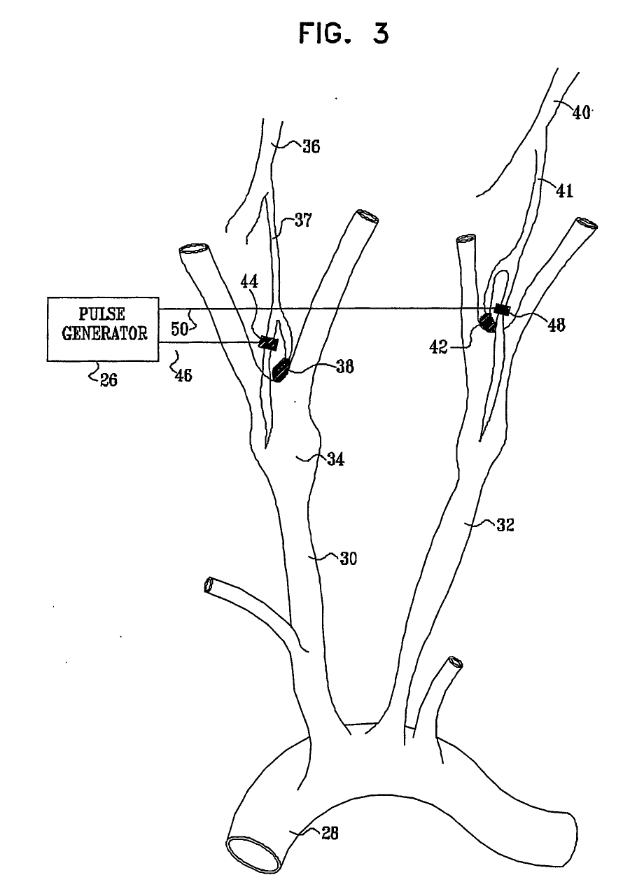

Reference is now made to FIG. 3, which is a fragmentary anatomic drawing. The description of FIG. 3 should be read in conjunction with FIG. 2. FIG. 3 illustrates neural and vascular structures which are relevant to an understanding of the arrangement 18 (FIG. 2), including an aortic arch 28, right common carotid artery 30, left common carotid artery 32, right carotid sinus 34, right glossopharyngeal nerve 36, right carotid body 38, left glossopharyngeal nerve 40, and left carotid body 42. An electrode 44 or plurality of electrodes 44 is attached or otherwise electrically coupled to the right glossopharyngeal nerve 36, and is connected to the pulse generator 26 by a lead 46. Preferably, the electrode 44 is attached to a branch of the right glossopharyngeal nerve 36, most preferably to the right carotid sinus nerve 37 at a site receiving sensory information from the right carotid sinus 34. Another electrode 48 or plurality of electrodes 48 is preferably applied contralaterally, i.e., to the left glossopharyngeal nerve 40, most preferably to the left carotid sinus nerve 41. The electrode 48 is connected by a lead 50 to a pulse generator, which can be the pulse generator 26, or a second pulse generator (not shown). In the latter case, the second pulse generator (not shown) is implanted in the same manner as the pulse generator 26, generally on the opposite side of the patient 22. The structure disclosed in U.S. Pat. No. 4,201,219 is suitable for the electrodes 44, 48.

The pulse generator 26 can conveniently be implanted in the vicinity of the clavicle, the mandible, or in other suitable positions, such as those known in the art for implantation of cardiac pacemakers.

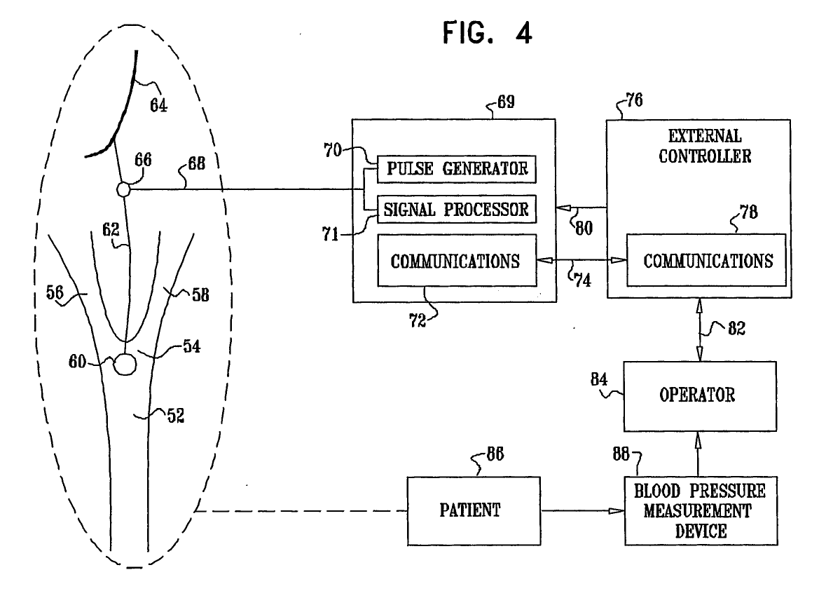

Reference is now made to FIG. 4, which is a schematic diagram illustrating the arrangement for blood pressure control shown in FIG. 2 in further detail. A carotid arterial system includes a common carotid artery 52, and its bifurcation 54 into an internal carotid artery 56 and an external carotid artery 58. A carotid sinus baroreceptor 60 is situated at the bifurcation 54, and transmits impulses over a carotid sinus nerve 62. The carotid sinus nerve 62 communicates with a larger branch of a glossopharyngeal nerve 64. A neurostimulation electrode 66 is preferably implanted on the carotid sinus nerve 62. The electrode 66 is attached by a lead 68 to a pulse generator 70 incorporated into an implanted unit 69. A communications module 72 of the implanted unit 69 receives instructions from and sends data to a communications module 78 of an external controller 76, which is not implanted in the patient. Preferably, but not necessarily, communication with the external controller 76 is performed over a wireless link 74. In some embodiments a module corresponding to the processor 24 (FIG. 2) can be incorporated in the external controller 76, in which case a firing rate or timing instruction is communicated to the pulse generator 70. In other embodiments the processor is integrated in the pulse generator 70, in which case patient blood pressure information is supplied by the external controller 76 to the communications module 72 of the pulse generator 70. The wireless link 74 may also be used for transmitting status information from the implanted unit 69 to the external controller 76.

The external controller 76 may also supply power over a wireless link 80 to the implanted unit 69, for example, by magnetic induction. The power may be used to support the operation of the implanted unit 69, and for recharging batteries (not shown) therein. The implanted unit 69 typically carries out a relatively simple task, which does not require large amount of signal processing. Its pulse discharge duty cycle is low, and thus power requirements are also low. Even without recharging, the implanted unit 69 can be expected to operate for months to years without the need for a battery replacement.

While only one electrode is shown in FIG. 4, it will be understood that the contralateral glossopharyngeal nerve may also be stimulated, using the pulse generator 70, or a second pulse generator (not shown), which is also controlled by the external controller 76. In a preferred embodiment, the electrode 66 comprises a monopolar electrode. Alternatively, the electrode 66 comprises bipolar or multipolar electrodes. In this latter case, two of the electrodes are preferably configured such that their applied current induces anterograde stimulation, while one or more of the other electrodes impose retrograde nerve block.

The external controller 76 is provided with a standard man-machine interface 82, such as a keypad and display, for use by an operator 84. The operator 84 obtains blood pressure data from a patient 86 using a standard blood pressure measurement device 88. Blood pressure data obtained in this manner are stored for a relatively long period of time in the external controller 76 or the pulse generator 70, and is referred to herein as static blood pressure. It is an advantage of this embodiment that instantaneous blood pressure need not be measured dynamically, and consequently the need to implant a blood pressure transducer is avoided.

Operation.

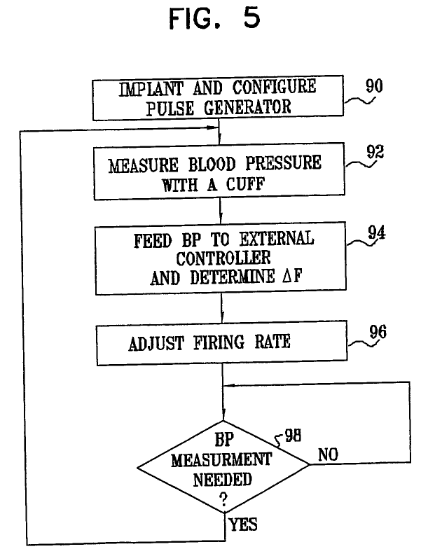

Reference is now made to FIG. 5, which is a flow chart illustrating the method of operation of the arrangement for blood pressure regulation that is illustrated in FIG. 4.

In initial step 90 the components of the arrangement 18 are applied to the patient 22. Stimulating electrodes are applied to the carotid sinus nerves and/or glossopharyngeal nerves of a patient using standard surgical techniques. A pulse generator is implanted and configured by an external controller. Baseline blood pressure information is obtained from the patient, and an initial firing rate is input into the pulse generator. The system is energized and begins operation.

At step 92 the patient's blood pressure is determined using standard blood pressure measuring equipment (such as a standard blood pressure cuff), and is subsequently inputted either manually or automatically into the external controller 76. At step 94 a computation is made to determine the appropriate firing rate of the type II baroreceptors in order to achieve a target blood pressure in the patient. This is done according to the function
ΔF=H(P_{measured}−P_{required}) (1)
where ΔF is the adjustment required to be made in the firing rate of the pulse generator; P_{measured} is the blood pressure of the patient that was determined in step 92; and P_{required} is the firing rate required to achieve a target blood pressure, which is determined from the response curve of the type II baroreceptors (FIG. 1B). The function H converts the resulting pressure differential into a firing rate according to the relationships shown in FIG. 1B. Alternatively or additionally, the function H is determined responsive to a mode of operation of the device, which is in turn typically determined responsive to clinical indications (e.g., history of heart failure, stroke, or hypertension). In a possible embodiment of the invention, the equation 1 is linear. However it is also possible to utilize non-linear transfer functions as well.

At step 96 the value ΔF is input into the pulse generator, and the pulse generator modifies its firing rate according to the formula
Fₙ=Fₙ₋₁+ΔF (2)
where Fₙ represents the firing rate of the pulse generator following its n^{th} adjustment, and Fₙ₋₁ represents the firing rate of the pulse generator immediately prior to its n^{th} adjustment. Appropriate limits are programmed into the pulse generator to prevent the firing rate from violating a predetermined safety range, as may be appropriate for a particular patient. The firing rate of the pulse generator is also constrained within the physiological range of the type II baroreceptors, typically 1-15 pulses per second, most preferably 1-6 pulses per second.

At delay step 98 a determination is made whether new blood pressure information is required to be obtained from the patient. A delay interval is established for each patient, based on his particular medical status and history. If the determination at delay step 98 is negative then control remains at delay step 98.

If the determination at delay step 98 is affirmative then control returns to step 92, and the process repeats.

### Second Embodiment

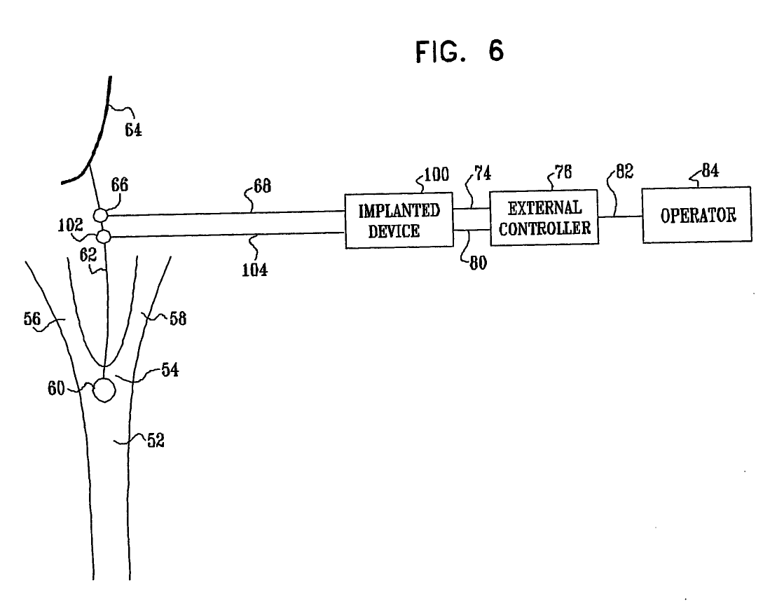

Reference is now made to FIG. 6, which is a schematic and block diagram of an arrangement for controlling blood pressure, which is constructed and operative in accordance with an alternate embodiment of the invention. The embodiment of FIG. 6 shares certain features with the embodiment of FIG. 4, but is more advanced. Like elements in FIG. 4 and FIG. 6 are given like reference numerals.

Using an estimate of the patient's blood pressure, based on type II baroreceptor activity, an implanted device 100 dynamically and automatically adapts its stimulation pulse rate to the patient's tonic blood pressure level. This feature allows for essentially autonomous operation. The implanted device 100 monitors the neural activity on the carotid sinus baroreceptor nerve in order to evaluate tonic blood pressure. In addition to the stimulating electrode 66, a sampling electrode 102 is placed on the carotid sinus nerve 62, and is connected to the implanted device 100 by a lead 104. The electrode 102 is responsive to nerve impulses that are transmitted via the carotid sinus nerve 62. Its structure is typically similar to the electrode 66. For some applications, the functionality as described with reference to the apparatus shown in FIG. 6 is alternatively realized by means of a multi-contact nerve electrode, in which some or all of the stimulation and sensing functionality is attained using common leads. As in the embodiment of FIG. 4, it will be understood that the arrangement is typically duplicated for the contralateral glossopharyngeal nerve, using the same or a different implanted device. As is explained in further detail hereinbelow, the implanted device 100 incorporates a processor to receive signals from the electrode 102, make the computations required to determine the appropriate firing rate to stimulate the glossopharyngeal nerve 64, and adjust the pulse rate of a signal delivered to the electrode 66. In some embodiments the electrode 66 and the electrode 102 can be placed on different nerves.

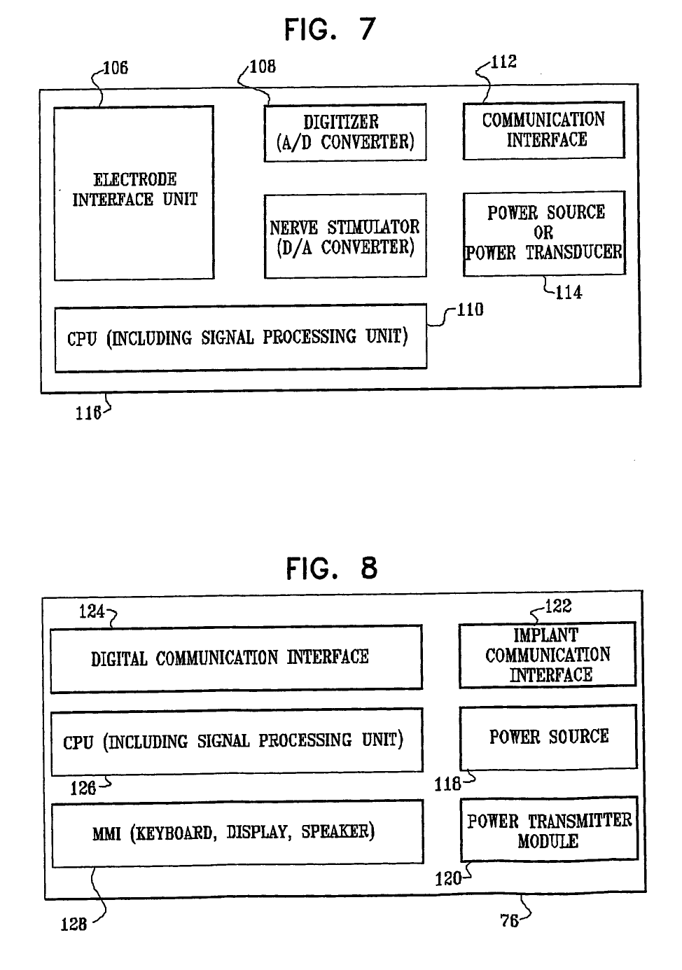

Reference is now made to FIG. 7, which is a detailed block diagram of the implanted device 100 (FIG. 6). The leads 68, 104 (FIG. 6) connect to the electrode interface unit 106. Signals received from the sensory electrode 102 are conditioned, and passed to a digitizer 108, which is a conventional analog-to-digital converter. A pulse generator 110 functions as a nerve stimulator. The pulse generator 110 includes a conventional digital-to-analog converter, the analog output of which is coupled to the electrode interface unit 106 for transmission on the lead 104 to the glossopharyngeal nerve 64 (FIG. 6). The implanted device 100 includes a communication interface 112 for communicating with the external controller 76 (FIG. 6). The implanted device 100 is powered by a power source 114, which may be a battery, and optionally can include an energy transducer for providing power or recharging the battery. For some applications, charging of the power source is realized through external charging means that include one or more of the following: kinetic charging means, acoustic (e.g., ultrasound) charging means, magnetic charging means, or electromagnetic charging means. The computation of the appropriate firing rate for the pulse generator 110 is performed by a central processing unit 116, which can include signal processing circuitry. The central processing unit 116 has an output connected to the pulse generator 110 and receives input from the digitizer 108, and is programmed to perform signal detection and processing. In one embodiment the central processing unit 116 is programmed to track a moving-average of integrated neural signal power, and to detect peaks. In other embodiments circuitry is provided to perform the integration and peak detection. Synchronization with the cardiac cycle facilitates accurate simulation of the physiologic baroreceptor discharge pattern. In some embodiments specialized signal processing circuitry, such as an application-specific integrated circuit (ASIC) may be used as the central processing unit 116.

Reference is now made to FIG. 8, which is a block diagram of the external controller 76 (FIG. 6). The external controller 76 is provided with a conventional power source 118, which can be a battery. A power transmitter module 120, such as an induction device, is used to transmit power over the link 80 (FIG. 6). A communication interface 122 exchanges data with the implanted device 100 (FIG. 6), using the wireless link 74. A digital communication interface 124 preferably enables direct coupling of the external controller to standard blood pressure measurement apparatus and/or to a personal computer (e.g., the physician's PC) to allow logging and analysis of treatment information. A central processing unit 126 is linked to the communication interface 122. The external controller 76 is provided with a conventional man-machine interface 128, which can include a keypad and a screen display. The man-machine interface 128 is utilized to input calibration parameters, such as the patient's particular type II baroreceptor activity data. The central processing unit 126 accepts this data, and prepares calibration parameters to be communicated to the implanted device 100 using the communication interface 122.

Referring again to FIG. 6, since the carotid sinus baroreceptor nerve is a neural tract, containing both type I and type II baroreceptor nerves, the implanted device 100 needs to discriminate the impulses of the two types of baroreceptors. This is preferably done by identifying dynamically silent periods of time, e.g., diastole, during which only type II discharges exist. Neural discharge signals that are received by the implanted device 100 during such dynamically silent periods are integrated to estimate tonic blood pressure. In a preferred embodiment, indications of systole and diastole are derived by analyzing the electrical signals traveling along the carotid sinus nerve. Systole, which is mechanically characterized by a fast rising and falling arterial pressure wave, can be identified by correspondingly fast changes in type I baroreceptor activity, i.e., activity at several tens of spikes per second. Diastole, by contrast, is identified by the absence of this high spike rate period, such that substantially the only activity measured is type II baroreceptor activity, i.e., activity less than about fifteen spikes per second. The spike rate during diastole, therefore, serves as an indicator of diastolic blood pressure. Based on a determination of the statistical relationships (e.g., mean, median, peak amplitudes, etc.) between arterial blood pressure and detected spike rates, the implanted device preferably identifies a time interval during which the discharge of type II baroreceptors is the sole contributor or essentially the sole contributor to the baroreceptor signals in the carotid sinus nerve. Responsive to identifying the time interval, the implanted device applies pulses to the carotid sinus nerve typically at less than 15 Hz, in order to simulate a higher diastolic blood pressure than actually exists, and, in response, induce a cardiovascular response which lowers blood pressure.

Advantageously, in this embodiment, the role of the external controller 76 is limited to initial or intermittent calibration of the implanted device 100, and for obtaining status information. The external blood pressure measurement device 88 (FIG. 4) is omitted in routine operation. Instead, the implanted device 100 relies for feedback control on its internal estimation of blood pressure, based upon afferent neural signals that are transmitted in the carotid sinus baroreceptor nerve.

Calibration.

A calibration procedure is typically required to train the implanted device 100 to correlate signals of the neural discharge pattern with actual blood pressure values measured with conventional techniques. As explained hereinabove, the relationship between blood pressure and type II baroreceptor discharge varies extremely slowly over time. No significant adaptation or resetting occurs for type II baroreceptors. Thus operation of the implanted device 100 in a patient is expected to be quite stable, and the calibration procedure may be performed infrequently, e.g., daily, weekly, or monthly. Advantageously, from the operator's perspective, calibration is similar to performing an ordinary blood pressure measurement, whereby input of the blood pressure measurement into the device initiates the calibration procedure.

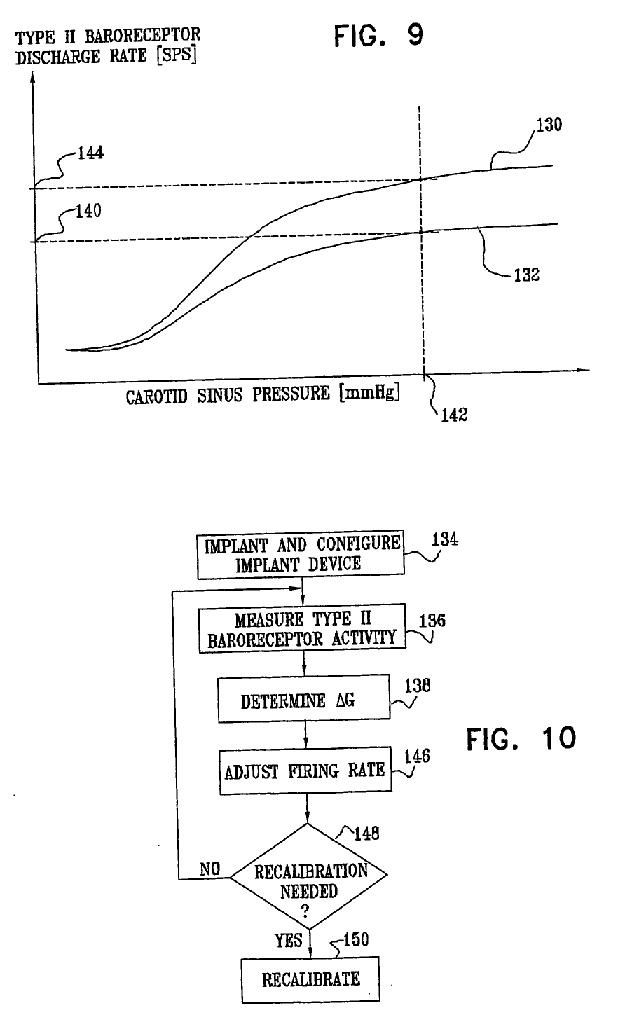

Reference is now made to FIG. 9, which illustrates plots of type II baroreceptor activity against carotid sinus pressure. A curve 130 represents physiological type II baroreceptor activity. A curve 132 represents type II baroreceptor in a typical hypertensive individual. It will be apparent that the type II baroreceptor response to blood pressure change in the hypertensive individual is blunted. In some embodiments the data of the curves 130, 132 are programmed into the external controller 76 (FIG. 6), which, using the central processing unit 126 (FIG. 8), prepares a table of firing rate correction data, using the differences between the curves 130, 132, and transmits the firing rate correction data to the implanted device 100 (FIG. 6). In other embodiments, the raw data of the curve 130 and the curve 132 are communicated by the external controller 76 to the implanted device 100, and a firing rate correction table is prepared by the central processing unit 116 (FIG. 7). Blood pressure measurements may also be input into the external controller 76, using the man-machine interface 128 (FIG. 8). Once the implanted device 100 is in operation, the type II baroreceptor activity characteristics of the particular patient may be determined, and the firing rate correction table adjusted accordingly.

It will be apparent to those skilled in the art that many techniques of storing firing rate correction data in a memory (not shown) of the central processing unit 126 or the central processing unit 116 can be used. For example, functional parameters describing the curves 130, 132 could be provided.

Operation.

Reference is now made to FIG. 10, which is a flow chart illustrating the method of operation of the arrangement for blood pressure regulation that is illustrated in FIGS. 6, 7, and 8.

In initial step 134, conventional surgical procedures are used for installing the implanted device 100 and attaching the electrodes 66, 102 to the glossopharyngeal nerve, preferably bilaterally. The external controller is initialized by utilizing generic baroreceptor activity data and type U baroreceptor activity information. Firing rate correction tables are prepared. The system is energized and begins operation.

At step 136 the patient's type II baroreceptor activity is determined by reading the signal obtained from the electrode 102. Then, at step 138 a lookup of the firing rate correction table is made, using the information obtained in step 136 and an adjustment factor calculated, which can be understood with reference to the following example. While the example is explained with reference to the graph of FIG. 9, it will be understood that data corresponding to the graph is typically stored in a table for convenient use by a processor.

Referring again to FIG. 9, in an example a value R₁ 140 may be read at step 136, and a carotid sinus pressure indicated by a point 142 can be inferred. The physiologic type II baroreceptor discharge rate corresponding to the point 142 is indicated by a value R₂ 144. A compensation ΔG in the firing rate of the pulse generator 110 is determined by subtracting the value 144 current firing rate from the corresponding entry in the firing rate correction table.
ΔG=R₂−R₁ (3)

Next, at step 146, the firing rate of the pulse generator is corrected according to the formula
Gₙ=Gₙ₋₁+ΔG (4)
where Gₙ represents the updated firing rate of the pulse generator 110 following its n^{th} adjustment, and Gₙ₋₁ is the firing rate determined in the prior iteration. Appropriate limits are programmed into the pulse generator 110 to prevent the firing rate from violating a predetermined safety range, as may be appropriate for a particular patient. The firing rate of the pulse generator is also typically constrained within the physiological range of the type II baroreceptors. The signal reaching the cardiovascular center of the brain stem thus may be considered to be a temporal summation of the patient's intrinsic type II baroreceptor impulses, and an extrinsic component supplied by the implanted device 100. It is noted that although spike activity along type I baroreceptor fibers is also affected by the artificially-applied pulses, this effect is generally very small, as the typical spike rate in the type I baroreceptor fibers is generally approximately one order of magnitude higher than the spike rate of the applied pulses. Moreover, since the artificially-applied pulses are typically applied when the type I baroreceptor fibers are generally silent (i.e., during systole), the ongoing estimations of systolic blood pressure in the patient are not greatly influenced by the operation of the device.

Control proceeds to decision step 148, where a test is made to determine if recalibration of the implanted device 100 is necessary. A typical criterion for recalibration is the expiration of a predetermined time interval. However, other criteria can also be used, for example, if the adjustment ΔG exceeds certain predefined parameters. Large excursions of the adjustment ΔG may indicate instability in the implanted device 100, or could indicate a change in the medical status of the patient. Either event could indicate the need for recalibration. In any case, periodic recalibration is typically desirable because of the continually varying nature of all living organisms. Thus, for example, if the patient's hypertension becomes less severe, then the compliance of the blood vessel walls in the carotid sinus may improve, and, consequently, the mechano-electrical transduction properties of the baroreceptors may undergo changes.

If the determination at decision step 148 is negative then control returns to step 136, and another iteration begins.

If the determination at decision step 148 is positive then control proceeds to step 150. The implanted device 100 is then recalibrated, as described above. Control then returns to step 136. In some embodiments of the method shown in FIG. 10, iterations occur with sufficient frequency to adjust the firing rate of the pulse generator 110 during different segments of the cardiac cycle.

Thus, using the techniques and apparatus described herein, it is seen that apparatus for treating or diagnosing a patient may perform one or more of the following:

(a) estimate diastolic and/or systolic blood pressure based on baroreceptor nerve signals, and set a stimulation parameter responsive thereto. For example, the rate and timing of stimulation of the carotid sinus nerve may be set based on the determined blood pressure.

(b) estimate diastolic blood pressure based on type II baroreceptor discharge.

(c) estimate systolic blood pressure based on type I baroreceptor discharge.

(d) identify one or more phases in the cardiac cycle based on type I and/or type II discharge, and stimulate responsive thereto.

(e) utilize intermittent external blood pressure measurements as inputs for calibration of measurements of type I and/or type II baroreceptor activity.

Preferably, each of these is accomplished substantially without an implanted mechanical blood pressure sensor (e.g., without using an implanted piezoelectric or capacitor-based pressure sensor). Instead, the only mechanical blood pressure measurements which are utilized preferably are performed relatively infrequently, e.g., less than every 12 hours, or, more preferably, less than once a day or once a week. Moreover, the sensing and stimulating functions are preferably, but not necessarily, performed at least in part using common electrodes.

In a preferred embodiment, methods and apparatus described herein for monitoring diastolic and/or systolic blood pressure are configured to operate in conjunction with a drug delivery device which, typically but not necessarily, delivers an antihypertensive medication. Advantageously, this overcomes one or more of the following problems typically associated with the frequent intake of antihypertensive medications:

(a) Patient non-compliance: The prescribed regimen of antihypertensive medication intake is often interrupted by factors that are dependant upon the patient For example, patients not infrequently forget to bring their pills when they go out, they forget having taken a dose and therefore take a second, unnecessary dose, or they feel fine and reason that they do not need to take a particular dose. A drug delivery device, such as is known in the art, operating in a closed loop with blood pressure measurement apparatus that implements techniques described herein avoids these substantial difficulties related to patient non-compliance.

(b) Dose mismatch: Neurological, humoral, and other factors determine a patient's basal blood pressure, and any of these may change over the course of days, leading to a mismatch between the actual cardiovascular status of the patient and the antihypertensive medication dosage. Integrating apparatus which regulates the delivery of the medication based on the values of blood pressure measured using techniques described herein overcomes this problem (e.g., based on values from the past hour, 12 hours, 24 hours, 48 hours, etc.).

It will be appreciated by persons skilled in the art that the present invention is not limited to what has been particularly shown and described hereinabove. Rather, the scope of the present invention includes both combinations and sub-combinations of the various features described hereinabove, as well as variations and modifications thereof that are not in the prior art which would occur to persons skilled in the art upon reading the foregoing description.

1. A system for neural stimulation for controlling cardiovascular function in a living body comprising:
an pulse generator for producing a pulsed electrical signal at a variable output rate, said pulse generator comprising:
a neurostimulation electrode;
a first lead, for conducting said pulsed electrical signal to said neurostimulation electrode; and
a first communications interface for receiving an external control signal, said output rate being responsive to said control signal;
wherein said output rate is within a range of activity of a baroreceptor.

2. The system according to claim 1, wherein said baroreceptor is a type II baroreceptor.

3. The system according to claim 1, wherein said baroreceptor is a type I baroreceptor.

4. The system according to claim 1, wherein said neurostimulation electrode is adapted to be attached to a nerve, and said nerve carries afferent baroreceptor impulses.

5. The system according to claim 4, wherein said nerve is a carotid sinus nerve branch of a glossopharyngeal nerve.

6. The system according to claim 1, wherein said range is from 5 pulses per second to 15 pulses per second.

7. The system according to claim 1, further comprising an external controller for generating said control signal.

8. The system according to claim 7, wherein said external controller comprises a second communications interface for transmitting said control signal to said first communications interface of said pulse generator via a wireless link.

9. The system according to claim 7, wherein said external controller comprises a man-machine interface for receiving a cardiovascular parameter therethrough.

10. The system according to claim 9, wherein said cardiovascular parameter is a blood pressure.

11. The system according to claim 10, wherein said blood pressure is a static blood pressure.

12. The system according to claim 9, wherein said cardiovascular parameter is transmitted in said control signal.

13. The system according to claim 9, wherein responsive to said cardiovascular parameter an adjustment to be made in said output rate is transmitted in said control signal.

14. The system according to claim 1, further comprising:
a sampling electrode;
a second lead, for conducting a sensory electrical signal from said sampling electrode to said pulse generator;
wherein said output rate is responsive to said sensory electrical signal.

15. The system according to claim 14, further comprising:
discrimination circuitry in said pulse generator for identifying information in said sensory electrical signal representing activity of a type II baroreceptor.

16. The system according to claim 15, wherein said output rate is responsive to said information.

17. The system according to claim 14, wherein said neurostimulation electrode and said sampling electrode are adapted to be attached to a nerve, and said nerve carries baroreceptor impulses.

18. The system according to claim 17, wherein said nerve is a carotid sinus nerve branch of a glossopharyngeal nerve.

19. The system according to claim 17, wherein said neurostimulation electrode and said sampling electrode are adapted to be attached to different nerves.

20. The system according to claim 14, wherein said sensory electrical signal is representative of an output of said baroreceptor.

21. The system according to claim 14, further comprising an external controller for generating said control signal.

22. The system according to claim 21, wherein said external controller comprises a second communications interface for transmitting said control signal to said first communications interface of said pulse generator via a wireless link.

23. The system according to claim 21, wherein said external controller comprises a man-machine interface for receiving calibration or operational information therethrough.

24. A method for controlling cardiovascular function in a living body comprising the steps of:
conducting a pulsatile signal to a nerve, said nerve carrying baroreceptor impulses at a stimulation rate that is within a range of activity of a type II baroreceptor;
measuring a value of a cardiovascular parameter in said body; and
adjusting said stimulation rate responsive to said value.

25. The method according to claim 24, wherein said cardiovascular parameter is a blood pressure.

26. The method according to claim 25, wherein said blood pressure is a diastolic blood pressure.

27. The method according to claim 24, wherein said cardiovascular parameter is a type II baroreceptor output signal.

28. The method according to claim 27, wherein said value is a rate of said type II baroreceptor output signal, and wherein said stimulation rate is a summation of said value and a compensatory value.

29. The method according to claim 24, wherein said nerve is a carotid sinus nerve branch of a glossopharyngeal nerve.

30. The method according to claim 24, wherein said stimulation rate is less than 15 pulses per second.
